(19) United States
(12) Patent Application Publication
(10) Pub. No.: US 2012/0122189 A1
(43) Pub. Date: May 17, 2012

(54) Lactobacillus Mutant

(76) Inventors: HSIA-CHING HSIAO, Taipei City (TW); TZU-MING PAN, Sijhih City (TW)

(21) Appl. No.: 12/946,817

(22) Filed: Nov. 15, 2010

(51) Int. Cl. C12N 1/20

(52) U.S. Cl. 435/252.9

(57) ABSTRACT

The present invention discloses a Lactobacillus mutant, which is Lactobacillus paracasei subsp. paracasei PAN 101 and deposited in Agricultural Research Culture Collection (NRRL) in Nov. 13, 2009, and the accession number is NRRL B-50335; Lactobacillus paracasei subsp. paracasei PAN 101 can produce L(+)-lactate, and the production of L(+)-lactate in Lactobacillus paracasei subsp. paracasei PAN 101 can achieve a maximum amount by culturing Lactobacillus paracasei subsp. paracasei PAN 101 under an appropriate culture condition; Lactobacillus paracasei subsp. paracasei PAN 101 has the effect of acid resistance, alkali resistance and immune regulation.

### BACKGROUND OF THE INVENTION

1. Field of the Invention

The present invention relates to a Lactobacillus mutant, and more particularly to a Lactobacillus mutant which can produce L(+)-lactate and has the effect of acid resistance, alkali resistance and immune regulation.

2. Description of the Prior Art

Lactobacillus is a kind of bacterium which can metabolize carbohydrate and produce more than 50% of lactic acid. Lactobacillus is one of the most important probiotics, and the mechanism of Lactobacillus in promoting the quality of flora in intestines might include: (1) produce organic acid and lower the pH value in the intestines; (2) compete with harmful bacteria on nutrients; (3) adhere on the epithelium of intestinal mucosa for decreasing the space of proliferation of harmful bacteria; and (4) produce antibacterial substances.

Besides increasing good bacteria in human body and enhancing immunity, Lactobacillus further possesses the following health functions:

(1) Antitumor effect: Lactobacillus pharmaceutics and fermented milk are proven to inhibit transplanted animal cancer and the proliferation of cancer cell caused by chemical material, and also can inhibit the cancer naturally happened in long-term feeding and breeding animals; some researches on human demonstrate that ingesting fermented milk can inhibit the happen of breast cancer and the recurrence of bladder cancer; the mechanism of Lactobacillus pharmaceutics and fermented milk is to regulate immune function and inhibit the production of carcinogenic substances by intestinal flora.

(2) Lowering blood cholesterol: the Masaai people in Asia ingest a large amount of fermented milk which contains high level of cholesterol, but the incidence of cardiovascular related diseases is very low; the mechanism of the fermented milk to lower cholesterol might be decrease the absorption effect of intestines, promote the discharge of liver, and inhibit the synthesis of some materials.

(3) Lowering blood pressure: certain peptides in fermented milk can inhibit angiotensin converting enzyme (ACE) so as to lower blood pressure.

The consumers pay attentions to health increasingly and there are more and more functional foods having the effect of health promoting. Probiotic products are the functional foods and have the function of promoting the digestion of foods, increasing the dietary value of foods, promoting the metabolic disorders of lactose intolerance, lowering cholesterol, and stimulating the activation of immune system. It can improve the floral quality in the intestines by ingesting the probiotics so as to promote the health of the host. After ingesting the probiotics, which will confront the challenges of the digestive system, such as gastric acid and bile salt, the survival probiotics can exert the functions. Furthermore, the probiotics should possess the appropriateness and the stability for manufacture, so as to be developed to effective probiotic products.

In view of this, it is necessary to provide a novel Lactobacillus mutant, which possesses better ability for producing acid, resisting acid, resisting bile salt, and regulating immune system, so as to be employed to product excellent probiotic products.

### SUMMARY OF THE INVENTION

In view of the above shortcomings of the prior art, the inventor of the present invention resorted to past experience, imagination, and creativity, performed experiments and researches repeatedly, and eventually devised the present invention, a Lactobacillus mutant.

The major objective of the present invention is to provide the Lactobacillus mutant, which possesses the ability for resisting acid, resisting bile salt, resisting alkaline, and regulating immune system, so as to be employed to product excellent probiotic products.

Another objective of the present invention is to provide the Lactobacillus mutant, which can effectively produce lactic acid and has good viability, and the production of lactic acid can reach a maximum amount under appropriate culture condition, so as to elevate the related function of Lactobacillus.

According to the above objective, the present invention provides a Lactobacillus mutant, which is Lactobacillus paracasei subsp. paracasei PAN 101 and deposited in Agricultural Research Culture Collection (NRRL) in Nov. 13, 2009, wherein the accession number of Lactobacillus paracasei subsp. paracasei PAN 101 is NRRL B-50335.

### BRIEF DESCRIPTION OF THE DRAWINGS

For a complete understanding of the aspects, structures and techniques of the invention, reference should be made to the following detailed description and accompanying drawings wherein:

FIG. 1 is a trend graph of the viability test of Lactobacillus paracasei subsp. paracasei PAN 101 and Lactobacillus plantarum NTU 102;

FIG. 2 is a trend graph of the basic growth curve of Lactobacillus paracasei subsp. paracasei PAN 101;

FIG. 3 is a trend graph of the growth condition of Lactobacillus paracasei subsp. paracasei PAN 101 under different culture temperature;

FIG. 4 is a trend graph of the growth condition of Lactobacillus paracasei subsp. paracasei PAN 101 under different culture pH value;

FIG. 5 is a trend graph of the growth condition of Lactobacillus paracasei subsp. paracasei PAN 101 with static culture and shaking culture respectively;

FIG. 6A˜FIG. 6C are graphs of the proportion of spleen cells containing MHC II^{Hi} molecular marker, CD86⁺ molecular marker and CD80⁺ molecular marker respectively;

FIG. 7 is a graph of the production rate of immunoglobulin G (IgG);

FIG. 8A is a graph of the proportion of cells containing CD3⁺CD4^{+ molecular marker in spleen and Peyer's patch respectively;}

FIG. 8B is a graph of the proportion of cells containing CD3⁺CD8⁺ molecular marker in spleen and Peyer's patch respectively; and

FIG. 8C is a graph of the proportion of cells containing B220⁺ molecular marker in spleen and Peyer's patch respectively.

### DETAILED DESCRIPTION OF THE PREFERRED EMBODIMENTS

To achieve the foregoing objectives and effects, the inventors screen Lactobacillus mutant from the mutants of Lactobacillus paracasei subsp. paracasei, thus achieving a Lactobacillus mutant of the present invention.

The Lactobacillus mutant provided by the present invention is Lactobacillus paracasei subsp. paracasei PAN 101 and deposited in Agricultural Research Culture Collection (NRRL) in Nov. 13, 2009, and the given accession number is NRRL B-50335.

The characteristics of Lactobacillus paracasei subsp. paracasei PAN 101 include: (1) gram-positive; (2) lacking catalase; (3) having the ability of curding; (4) having the ability of acid resistance, alkaline resistance and bile salt resistance; (5) facultative heterogeneous fermentation; (6) producing L(+)-lactate; and (7) having excellent ability of immune regulation. Furthermore, Lactobacillus paracasei subsp. paracasei PAN 101 of the present invention is screened from the stool of an infant. The basic culture medium for Lactobacillus paracasei subsp. paracasei PAN 101 is MRS medium, wherein the best culture temperature is 30° C., the best culture time is 24 hours, the best culture pH value is 6.5, the best culture pressure is 1 atm, and Lactobacillus paracasei subsp. paracasei PAN 101 needs microaerophilic growth. Additionally, a culture manner of Lactobacillus paracasei subsp. paracasei PAN 101 can be a static culture or a shaking culture, which both can provide high quantity of production amount of lactic acid.

The method for testing the viability of Lactobacillus paracasei subsp. paracasei PAN 101 is following: serially diluting the bacteria from frozen tubes or bacterial powder; spreading the bacteria on the MRS medium; culturing the bacteria under 30 for 24˜48 hours and forming white-circular colonies. The preservation manner of Lactobacillus paracasei subsp. paracasei PAN 101 is to be stored in 30% of glycerol or stored with dry bacteria powder. For short-term preservation, the bacteria should be stored under 4° C.; for long-term preservation, the bacteria should be stored under −20° C.

In order to prove that Lactobacillus paracasei subsp. paracasei PAN 101 provided by the present invention possesses the above characteristics, the following analyzes and compares the functions and properties of Lactobacillus paracasei subsp. paracasei PAN 101 with several experiments.

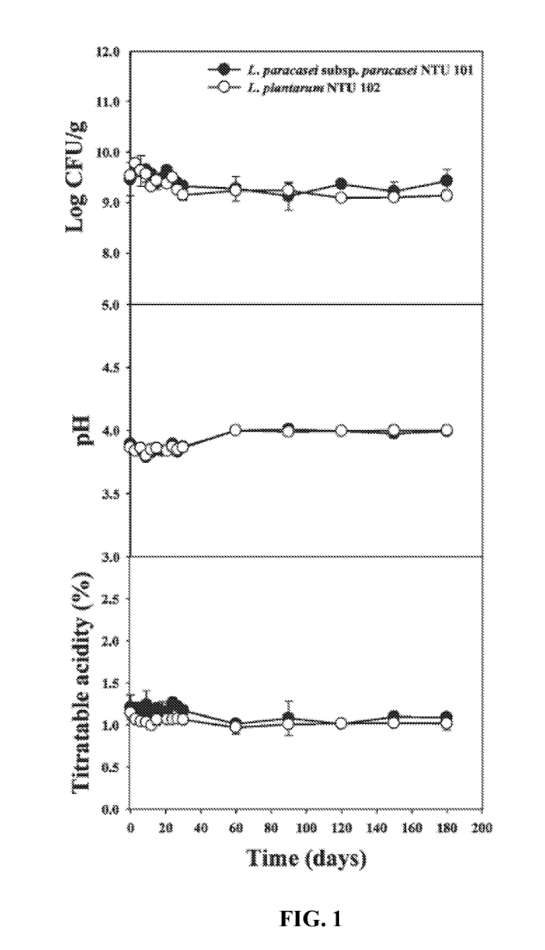

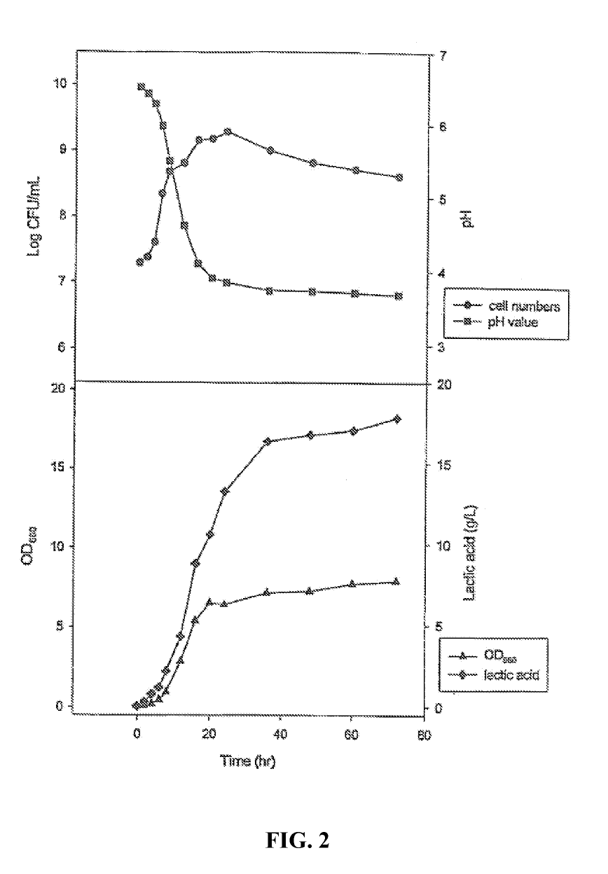

Referring to FIG. 1, which is a trend graph of the viability test of Lactobacillus paracasei subsp. paracasei PAN 101 and Lactobacillus plantarum NTU 102. The black solid circles present Lactobacillus paracasei subsp. paracasei PAN 101, and the white solid circles present Lactobacillus plantarum NTU 102; the horizontal axis presents the preservation time (days); vertical axis from top to bottom present viable count (Log CFU/g), pH value and titratable acidity (%). The results demonstrate that after the preservation of both species of bacteria under 4° C. for 180 days, the viable count, the pH value and the titratable acidity does not decay in both Lactobacillus paracasei subsp. paracasei PAN 101 and Lactobacillus plantarum NTU 102.

Referring to FIG. 2, which is a trend graph of the basic growth curve of Lactobacillus paracasei subsp. paracasei PAN 101. The horizontal axis presents the culture time (hours); the vertical axis in the upper graph presents viable count (Log CFU/g) and pH value; the vertical axis in the lower graph presents OD600 and the concentration of lactic acid (g/L). The results demonstrate that the viable count of Lactobacillus paracasei subsp. paracasei PAN 101 can increase with culture time, and the production amount of lactic acid also increase with culture time.

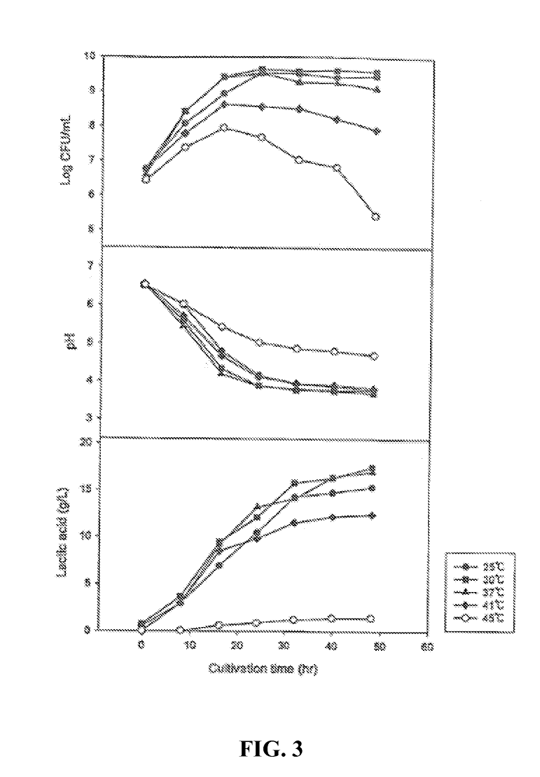

Referring to FIG. 3, which is a trend graph of the growth condition of Lactobacillus paracasei subsp. paracasei PAN 101 under different culture temperature. The horizontal axis presents the culture time (hours); the vertical axis in the upper graph presents viable count (Log CFU/g); the vertical axis in the middle graph presents pH value; the vertical axis in the lower graph presents the production amount of lactic acid (g/L). The results demonstrate that the best culture temperature for Lactobacillus paracasei subsp. paracasei PAN 101 is 30° C.

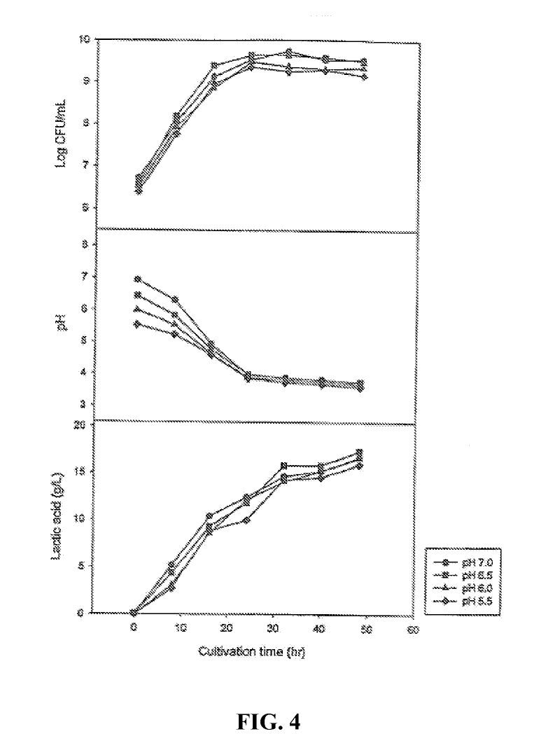

Referring to FIG. 4, which is a trend graph of the growth condition of Lactobacillus paracasei subsp. paracasei PAN 101 under different culture pH value. The horizontal axis presents the culture time (hours); the vertical axis in the upper graph presents viable count (Log CFU/g); the vertical axis in the middle graph presents pH value; the vertical axis in the lower graph presents the production amount of lactic acid (g/L). The results demonstrate that the best culture pH value for Lactobacillus paracasei subsp. paracasei PAN 101 is 6.5.

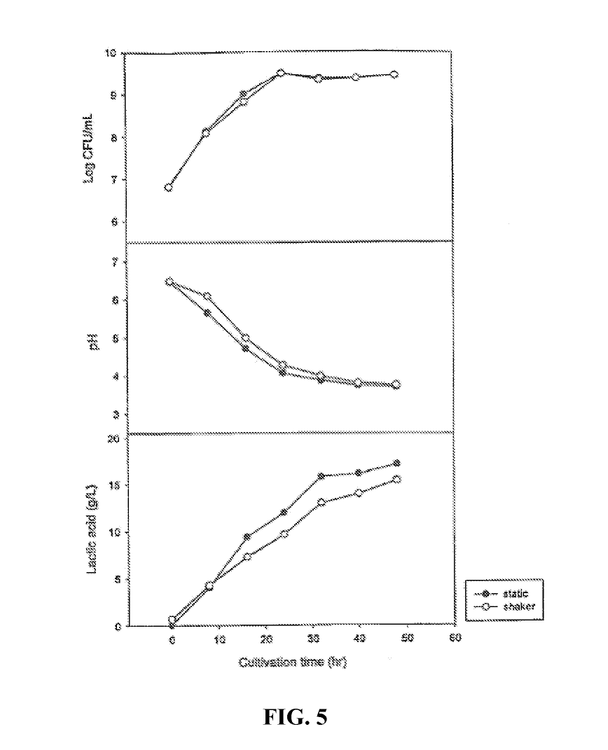

Referring to FIG. 5, which is a trend graph of the growth condition of Lactobacillus paracasei subsp. paracasei PAN 101 with static culture and shaking culture respectively. The horizontal axis presents the culture time (hours); the vertical axis in the upper graph presents viable count (Log CFU/g); the vertical axis in the middle graph presents pH value; the vertical axis in the lower graph presents the production amount of lactic acid (g/L). The results demonstrate that the viable count and the pH value in both culture manner are almost the same, but the static culture can obtain more L(+)-lactic acid (17.54 g/L) in 48 hours.

Referring to the following table 1, which illustrates the growth condition and the production amount of Lactobacillus paracasei subsp. paracasei PAN 101 cultured with MRS medium added with different carbon sources. The results demonstrate that Lactobacillus paracasei subsp. paracasei PAN 101 can utilize several kinds of carbon sources for producing well amount of lactic acid after 48 hours of culture. Wherein, Lactobacillus paracasei subsp. paracasei PAN 101 can utilize Glucose and D-mannitol for producing the maximum amount of lactic acid.

TABLE 1

| | | | Production |
|---|---|---|---|
| Carbon | viable count | pH | amount of lactic |
| source | (Log CFU/mL) | value | acid (g/L) |
| Glucose | 9.43 | 3.73 | 17.48 |
| Galactose | 9.33 | 3.70 | 11.33 |
| D-ribose | 9.54 | 4.07 | 7.25 |
| Xylose | 8.94 | 6.37 | 0.40 |
| Fructose | 8.20 | 3.75 | 14.00 |
| α-Lactose | 9.26 | 3.87 | 11.64 |
| Maltose | 9.45 | 4.16 | 8.55 |
| Sucrose | 9.01 | 3.78 | 13.90 |
| Trehalose | 9.04 | 3.79 | 13.26 |
| Raffinose | 8.78 | 5.23 | 1.80 |
| myo-Inositol | 8.89 | 6.48 | 0.41 |
| Sorbitol | 9.65 | 4.15 | 7.49 |
| D-mannitol | 9.44 | 3.81 | 16.21 |
| Citric acid | 7.05 | 6.41 | 0.28 |
| Dextrin | 9.38 | 5.35 | 0.86 |
| Strach | 9.24 | 5.82 | 0.30 |
| Molasses | 9.70 | 4.50 | 6.02 |

Referring to the following table 2, which illustrates the growth condition and the production amount of Lactobacillus paracasei subsp. paracasei PAN 101 cultured with MRS medium added with different nitrogen sources. The results demonstrate that Lactobacillus paracasei subsp. paracasei PAN 101 can utilize several kinds of nitrogen sources for producing well amount of lactic acid after 48 hours of culture. Wherein, Lactobacillus paracasei subsp. paracasei PAN 101 can utilize Yeast extract for producing the maximum amount of lactic acid.

TABLE 2

| | viable count | | Production amount |
|---|---|---|---|
| Nitrogen source | (Log CFU/mL) | pH value | of lactic acid (g/L) |
| Yeast extract | 8.14 | 3.54 | 8.29 |
| Beef extract | 8.89 | 4.22 | 2.74 |
| Peptone | 8.95 | 3.74 | 5.91 |
| Soytone | 8.30 | 3.90 | 5.82 |
| Tryptose | 8.84 | 3.87 | 4.45 |
| Corn-steep liquor | 9.14 | 4.14 | 4.11 |
| Casein | 8.27 | 4.68 | 1.77 |
| Urea | 6.89 | 5.96 | 0.02 |
| Ammonium citrate | 7.09 | 6.04 | 0.08 |
| Ammonium sulfate | 6.69 | 5.84 | 0.07 |

Referring to the following table 3, which illustrates the growth condition and the production amount of Lactobacillus paracasei subsp. paracasei PAN 101 cultured with different substrates. The results demonstrate that Lactobacillus paracasei subsp. paracasei PAN 101 can be cultured with several kinds of substrates for producing well amount of lactic acid after 48 hours of culture. Wherein, Lactobacillus paracasei subsp. paracasei PAN 101 cultured with milk for 48 hours can produce the maximum amount of lactic acid.

TABLE 3

| | viable count | | Production amount |
|---|---|---|---|
| substrate | (Log CFU/mL) | pH value | of lactic acid (g/L) |
| Soy beans | 9.00 | 4.97 | 0.03 |
| Mung beans | 8.29 | 6.31 | 0.01 |
| Adzuki beans | 8.27 | 6.17 | 0.01 |
| Job's tears | 7.33 | 5.52 | 0.01 |
| Milk | 9.07 | 3.58 | 0.60 |

Subsequently, compare the ability of acid resistance of Lactobacillus paracasei subsp. paracasei PAN 101 with other Lactobacillus species. Referring to the following table 4, I5-12, H5-1, A6-9, CCRC 11847, CCRC 10695, and CCRC 14026 are all lactobacilli. After culturing the above lactobacilli under pH 2.5 for 1 hour, except for H5-1 and CCRC 11847, the bacterial counts of other lactobacilli only decrease a little and have the viability about 70%˜80%; after 2 hours, the bacterial counts of I5-12, A6-9 and Lactobacillus paracasei subsp. paracasei PAN 101 still keeps on 10⁶ CFU/mL; after 4 hours, H5-1 present no colony, and the other lactobacilli have the bacterial counts about 10³˜10⁴ CFU/mL. It will be seen from this that Lactobacillus paracasei subsp. paracasei PAN 101 has the ability of acid resistance.

TABLE 4

| Lactobacillus | Time (hr) | | | | | |
|---|---|---|---|---|---|---|
| species | 0 | 0.5 | 1 | 2 | 3 | 4 |
| 15-12 | 6.65 | 6.60 | 6.36 | 6.28 | 5.48 | 4.29 |
| H5-1 | 6.73 | 6.54 | 5.77 | 4.53 | 3.85 | 0.00 |
| A6-9 | 6.79 | 6.71 | 6.45 | 6.33 | 5.90 | 4.34 |
| Lactobacillus | 7.21 | 7.05 | 7.02 | 6.52 | 4.12 | 3.25 |
| paracasei | | | | | | |
| subsp. | | | | | | |
| paracasei | | | | | | |
| PAN 101 | | | | | | |
| CCRC 11847 | 6.91 | 6.22 | 5.64 | 5.37 | 4.66 | 3.30 |
| CCRC 10695 | 6.68 | 6.53 | 6.38 | 5.76 | 5.11 | 3.03 |
| CCRC 14026 | 6.73 | 6.60 | 6.38 | 5.00 | 4.72 | 3.03 |

In order to test the ability of bile salt resistance, Lactobacillus paracasei subsp. paracasei PAN 101 and other Lactobacillus species are cultured with MRS broth medium added with 0.3% of oxgall, and these bacteria are tested for the required time to reach the absorbance value of 0.3, so as to compare the ability of bile salt resistance between each Lactobacillus species. Referring to the following table 5, the results demonstrate that the growths of all Lactobacillus species are inhibited by bile salt; the times to reach the absorbance value of 0.3 in H5-1 and CCRC 14026 are delayed more than 1 hour related to their control, thus H5-1 and CCRC 14026 are sensitive to bile salt; the times to reach the absorbance value of 0.3 in I5-2, A6-9, CCRC 11847, and CCRC 10695 are delayed for 59.6 min, 49.6 min, 42.5 min, and 51.8 min related to their control respectively, thus I5-2, A6-9, CCRC 11847, and CCRC 10695 have low tolerance to bile salt; the times to reach the absorbance value of 0.3 in Lactobacillus paracasei subsp. paracasei PAN 101 is only delayed for 27.5 min related to control, thus Lactobacillus paracasei subsp. paracasei PAN 101 has tolerance to bile salt.

TABLE 5

| | | MRS broth + |
|---|---|---|
| Lactobacillus | MRS broth | 0.3% oxgall |
| species | (control) (min) | (min) |
| I5-12 | 264.8 | 324.4 |
| H5-1 | 291.2 | 366.4 |
| A6-9 | 266.6 | 316.2 |
| Lactobacillus | 247.3 | 274.8 |
| paracasei subsp. | | |
| paracasei PAN | | |
| 101 | | |
| CCRC 11847 | 251.9 | 294.4 |
| CCRC 10695 | 293.2 | 345.0 |
| CCRC 14026 | 229.4 | 305.1 |

Subsequently, immune regulation related functions of Lactobacillus paracasei subsp. paracasei PAN 101 are tested by several experiments and analyses. The experiments of the present invention utilize mice as the experimental animal, and the mice are fed with Lactobacillus paracasei subsp. paracasei PAN 101 for analyzing the proportion of cells containing specific immune related molecular markers.

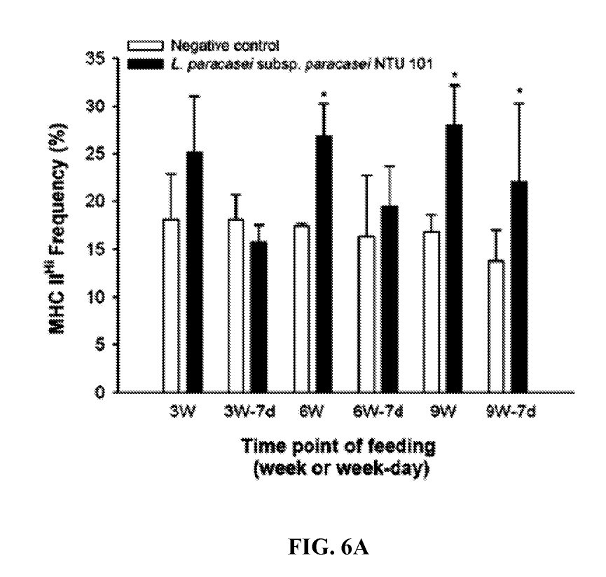

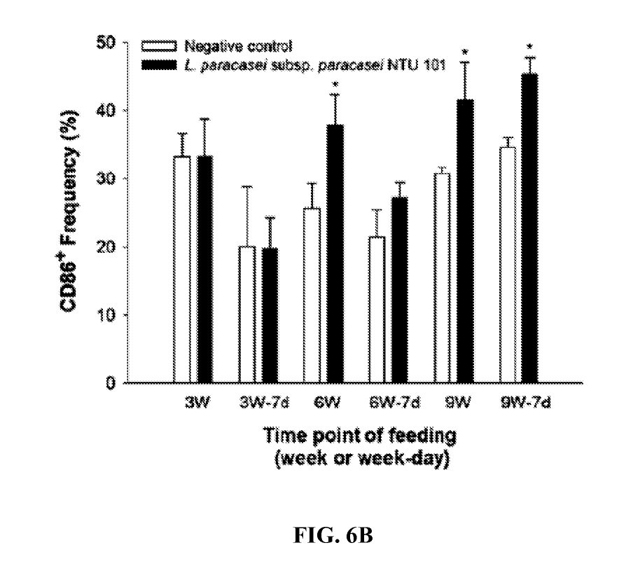

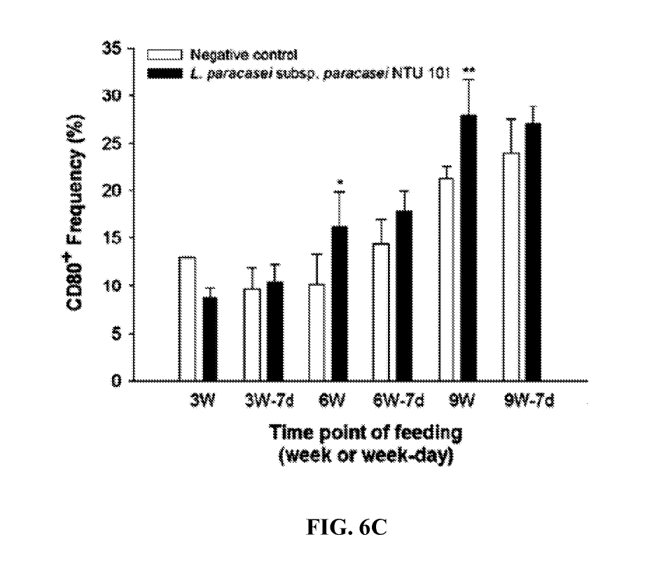

Referring to FIG. 6A˜FIG. 6C, which are graphs of the proportion of spleen cells containing MHC II^{Hi} molecular marker, CD86⁺ molecular marker and CD80⁺ molecular marker respectively. The results demonstrate that regardless of any feeding time, the mice fed with Lactobacillus paracasei subsp. paracasei PAN 101 can produce more spleen cells containing MHC II^{Hi} molecular marker, CD86⁺ molecular marker and CD80⁺ molecular marker related to control.

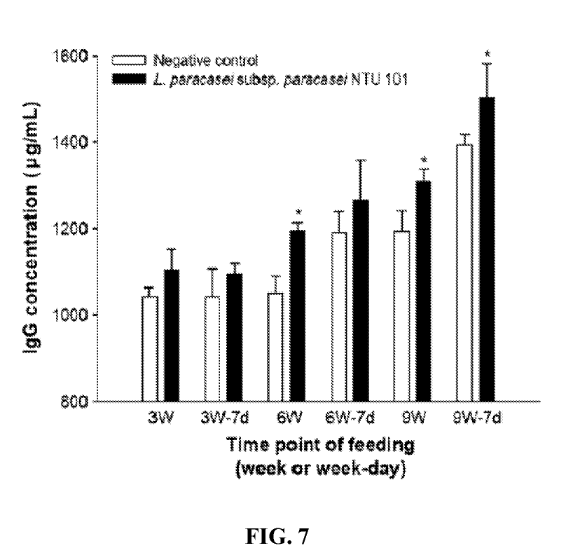

Referring to FIG. 7, which is a graph of the production rate of immunoglobulin G (IgG). The results demonstrate that regardless of any feeding time, the mice fed with Lactobacillus paracasei subsp. paracasei PAN 101 can produce more IgG related to control.

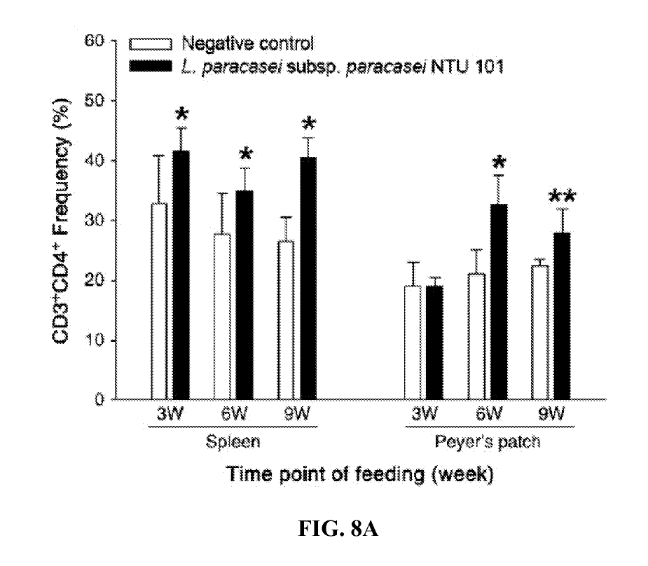

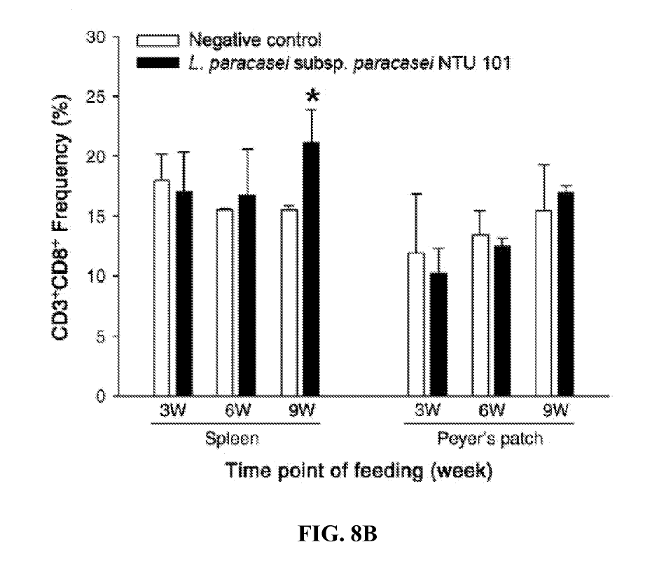

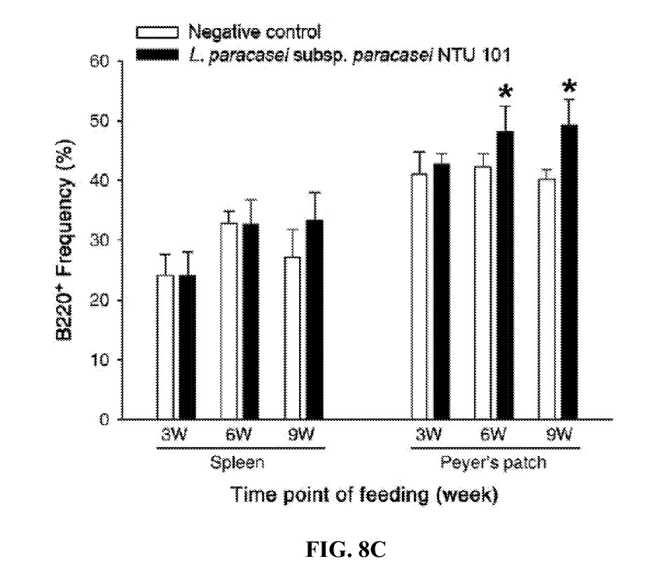

Referring to FIG. 8A˜FIG. 8C, wherein FIG. 8A is a graph of the proportion of cells containing CD3⁺CD4⁺ molecular marker in spleen and Peyer's patch respectively, FIG. 8B is a graph of the proportion of cells containing CD3⁺CD8⁺ molecular marker in spleen and Peyer's patch respectively, and FIG. 8C is a graph of the proportion of cells containing B220⁺ molecular marker in spleen and Peyer's patch respectively. The results demonstrate that regardless of spleen or Peyer's patch, and regardless of any feeding time, the mice fed with Lactobacillus paracasei subsp. paracasei PAN 101 can produce more cells containing CD3⁺CD4⁺ molecular marker, CD3⁺CD8⁺ molecular marker and B220⁺ molecular marker related to control.

From the above experiments, it can be proven that the mice fed with Lactobacillus paracasei subsp. paracasei PAN 101 can produce more amount of immune related cells, and it indicate that Lactobacillus paracasei subsp. paracasei PAN 101 indeed has the ability of increasing the production of immune cells, that is to say, Lactobacillus paracasei subsp. paracasei PAN 101 has the function of immune regulation.

By the detailed description of the overall structure and technical content of the present invention, the following advantages of the present invention can be derived:
1. Lactobacillus paracasei subsp. paracasei PAN 101 provided by the present invention can effectively produce lactic acid and has excellent viability, and Lactobacillus paracasei subsp. paracasei PAN 101 can produce lactic acid to a maximum amount under appropriate culture conditions, so as to elevate the related functions of Lactobacillus.
2. Lactobacillus paracasei subsp. paracasei PAN 101 provided by the present invention can effectively utilize several kinds of carbon sources, nitrogen sources and culture substrate for producing lactic acid, so as to elevate the related functions of Lactobacillus.
3. Lactobacillus paracasei subsp. paracasei PAN 101 provided by the present invention possesses the ability for resisting acid, resisting bile salt, resisting alkaline, and regulating immune system, so as to be employed to product excellent probiotic products.

It should be understood that the embodiments of the present invention described herein are merely illustrative of the technical concepts and features of the present invention and are not meant to limit the scope of the invention. Those skilled in the art, after reading the present disclosure, will know how to practice the invention. Various variations or modifications can be made without departing from the spirit of the invention. All such equivalent variations and modifications are intended to be included within the scope of the invention.

As a result of continued thinking about the invention and modifications, the inventors finally work out the designs of the present invention that has many advantages as described above. The present invention meets the requirements for an invention patent, and the application for a patent is duly filed accordingly. It is expected that the invention could be examined at an early date and granted so as to protect the rights of the inventors.

What is claimed is:

1. A Lactobacillus mutant, which is Lactobacillus paracasei subsp. paracasei PAN 101 and deposited in Agricultural Research Culture Collection (NRRL) in Nov. 13, 2009, wherein the accession number of Lactobacillus paracasei subsp. paracasei PAN 101 is NRRL B-50335.

2. The Lactobacillus mutant according to claim 1, wherein Lactobacillus paracasei subsp. paracasei PAN 101 can produce L(+)-lactate.

3. The Lactobacillus mutant according to claim 1, wherein Lactobacillus paracasei subsp. paracasei PAN 101 has the effect of acid resistance, alkali resistance and immune regulation.

4. The Lactobacillus mutant according to claim 1, wherein the best culture temperature is 30° C.

5. The Lactobacillus mutant according to claim 1, wherein the best culture pH value is 6.5.

6. The Lactobacillus mutant according to claim 1, wherein the best culture pressure is 1 atm (1 atmosphere pressure).

7. The Lactobacillus mutant according to claim 1, wherein a culture manner of Lactobacillus paracasei subsp. paracasei PAN 101 is selected from the group consisting of static culture and shaking culture.

8. The Lactobacillus mutant according to claim 1, wherein a culture medium for culturing Lactobacillus paracasei subsp. paracasei PAN 101 is able to contain at least one carbon source, which is selected from the group consisting of: Glucose, Galactose, D-ribose, Xylose, Fructose, α-Lactose, Maltose, Sucrose, Trehalose, Raffinose, myo-Inositol, Sorbitol, D-mannitol, Citric acid, Dextrin, Starch, and Molasses.

9. The Lactobacillus mutant according to claim 1, wherein a culture medium for culturing Lactobacillus paracasei subsp. paracasei PAN 101 is able to contain at least one nitrogen source, which is selected from the group consisting of: Yeast extract, Beef extract, Peptone, Soytone, Tryptose, Corn-steep liquor, Casein, Urea, Ammonium citrate, and Ammonium sulfate.
